(19) United States
(12) Patent Application Publication
(10) Pub. No.: US 2021/0330265 A1
(43) Pub. Date: Oct. 28, 2021

(54) SUPPORTING DEVICE FOR SUPPORTING A BODY PART OF A USER

(71) Applicant: FortHi GmbH, Lichtenfels (DE)

(72) Inventors: Manuel Franz, Wörrstadt (DE); Tobias Winkler, Dresden (DE); Christian Daniel Walter, Bad Kreuznach (DE)

(21) Appl. No.: 17/279,819

(22) PCT Filed: Sep. 27, 2019
(86) PCT No.: PCT/EP2019/076233

§ 371 Date: Mar. 25, 2021

(30) Foreign Application Priority Data
Sep. 27, 2018 (DE) 10 2018 007 850.8
Mar. 5, 2019 (DE) 10 2019 105 532.6

(51) Int. Cl. A61B 5/00; A61B 5/0205; A61F 7/03; A61F 7/00

(52) U.S. Cl. CPC A61B 5/6891; A61B 5/0205; A61B 5/0004; A61B 5/0022; A61B 5/7455; A61F 2007/0045; A61B 5/7405; A61F 7/03; A61F 7/007; A61B 2560/0425; A61F 2007/0036; A61B 5/742

(57) ABSTRACT

Support device (1) for supporting at least one body part of a user at least in portions, optionally completely, comprising a support body (2), which comprises at least one support region (3) for supporting at least one body part of a user at least in portions, optionally completely, wherein at least the support region (3) has, at least in portions, optionally completely, a geometric-structural design that is individually configured on the basis of the data describing at least one body part of a user that is to be supported on the support region (3) at least in portions, optionally completely.

### CROSS REFERENCE TO RELATED APPLICATIONS

The present specification is a National Phase Entry of International Application No. PCT/EP2019/076233 filed Sep. 27, 2019 and entitled “Supporting Device For Supporting a Body Part of a User” which, itself, claims priority to DE 10 2018 007 850.8 filed Sep. 27, 2018 and DE 2019 105 532.6 filed Mar. 5, 2019, each of which are incorporated by reference herein in their entireties.

### FIELD

The present specification relates to a support device for supporting at least one body part of a user at least in portions, optionally completely, comprising a support body, which comprises at least one support region for supporting at least one body part of a user at least in portions, optionally completely.

### BACKGROUND

Support devices are known per se from the prior art in different configurations.

A known embodiment of a corresponding support device comprises a support body, which comprises at least one support region for supporting at least one body part, e.g. a limb, of a user at least in portions, optionally completely.

The main function of a corresponding support device consists in supporting a body part supported on a corresponding support region of a support body of the support device. In this case, the body part is intended to be supported on the support region in a position that is as relaxed as possible, specifically to that body part. This may be expedient for relieving tension or influencing the internal tension (tone) of the muscles, optionally as part of therapy.

In this connection, there is a need to improve known support devices since the support bodies associated therewith are typically largely standardised moulded bodies, by means of which user-specific, optimised support of a relevant body can only be provided to a limited extent owing to the large number of anatomically different body parts, even when considering just one particular type of body part, such as a hand.

### SUMMARY

The object of the embodiments described herein is to provide a support device that is improved compared with the above for supporting a limb of a user.

The object is achieved by a support device according to claim 1. The claims that are dependent thereon relate to possible embodiments of the support device.

The support device described herein in general serves to support at least one body part of a user at least in portions, optionally completely. Wherever the term “user” is used below, this of course includes all genders.

The term “support” can be understood to mean mounting at least one body part or at least one portion of a body part. The support device can therefore be referred to as or considered to be a mounting device. The support body associated with the support device can accordingly be referred to as or considered to be a mounting body. The support region to be assigned to the support body can accordingly be referred to as or considered to be a mounting region.

The term “body part” can be understood both as a body part and as a portion of a body part. An example of a body part is the torso or a limb, and an example of a portion of a body part is a torso portion or a limb portion.

The term “limb” can be understood both as a complete limb (extremity) and as a limb portion of a superordinate limb. An example of a limb is therefore an arm or a leg, and an example of a limb portion is a hand or a foot.

A hand or a foot may, however, also be an example of a limb, and an example of a limb portion may therefore also be a finger or a toe.

It is clear from the above comments that the support device can be configured in particular for supporting a limb of an upper extremity of a user, in particular a hand, a portion of the hand, an arm or a portion of the arm, or for supporting a limb of a lower extremity of a user, in particular a foot, a portion of the foot, a leg or a portion of the leg.

In principle, in connection with the support device, body parts, i.e. in particular limbs, or portions of body parts, i.e. in particular portions of limbs, of humans or the human body are referred to; however, body parts or portions of body parts of an animal or animal body are also conceivable.

As indicated, the support device comprises a support body. The support body, which, as mentioned, can also be referred to as or considered to be a mounting body, comprises at least one support region, which, as mentioned, can also be referred to as or considered to be a mounting region, for supporting at least one body part, i.e. a limb, for example, of a user at least in portions, optionally completely. The support region is therefore understood to be the region of the support device on which a user (directly) supports a body part when the support device is used as intended. The support region therefore constitutes the actual region of the support device on which a user supports or mounts a body part when the support device is used as intended. The support region typically forms at least one part of the (exposed) surface of the support device.

The support device therefore substantially consists of an e.g. block-like or block-shaped support body, which comprises at least one surface which has, at least in portions, optionally completely, three-dimensional contours or structures that are individually configured in respect of a particular body part of a particular user. These three-dimensional contours or structures are typically formed by the support region in this case.

The dimensions of the support device, the support body and the support region are typically adapted to the dimensions of the body part to be supported in each case. When the support device is used as intended, the support region can therefore be completely covered by the body part being supported in each case.

The support device described herein is characterised in that at least the support region has, at least in portions, optionally completely, a geometric-structural design that is individually configured on the basis of the data describing at least one body part of a user that is to be supported on the support region at least in portions, optionally completely. The support region therefore has an individually configured geometric-structural design and thus has a high degree of customisation. In particular, the support region, and optionally also the support body and thus also the support device, may be a component that is individually configured or individually configurable for a particular body part of a particular user.

The individually configured geometric-structural design of the support region, with this in particular being understood to be particular contours or structures of the surface of the support region, has been produced on the basis of the data describing at least one body part of a particular user that is to be supported on the support region at least in portions, optionally completely, when used as intended. The support region therefore typically constitutes a (negative) reproduction, individually configured in a user-specific manner, of a body part of a particular user that is to be supported on the support region. This allows for a fit of the support region that is individually configured in a user-specific manner or a manner specific to a body part of the user and is thus highly precise, this fit allowing the relevant body part to be supported on the support region in a relaxing or relaxed position specific to this body part.

Supporting a body part in a relaxing or relaxed position specific to this body part can both have a relaxing effect on the relevant body part as well as on other body parts adjacent to this body part, such that the user's whole body can be brought into a relaxing or relaxed state when using the support device. In addition to the mere relaxing effect or as a consequence thereof, where necessary, therapeutic effects can also be brought about, since supporting the body part in a relaxing or relaxed position specific to this body part can e.g. have a therapeutic effect on the blood flow and/or oxygen supply to the relevant body part. Likewise, the effect of reducing swelling could also be demonstrated. Positive effects could be identified in particular in rheumatic body parts or limbs and/or in e.g. spastic body parts or limbs due to a stroke. The increase in the internal tension (tone) of the muscles of the relevant body part, associated with spasticity, could be reduced (significantly in some cases) by (regular) use of the support device.

The user can use the support device in particular without any help from a third party; therefore, the user can independently carry out therapeutic measures, i.e. for regulating tone, pain therapy, etc., and prepare themselves for corresponding therapeutic measures in the sense of self-help by using the support device as intended.

Using the example of a spastic hand, the support device, which, in this respect, can be referred to as or considered to be a customised support for the heel of the hand for providing therapy to persons with a spastic or cramped hand, makes it possible for the hand to rest on the support or support device in a loosened state. By supporting the cramped hand on the customised support surface, a state of relaxation is achieved. The hand is reproduced and positioned by the shape of the customised support for the heel of the hand with a precise fit.

Overall, an improved support device is provided for supporting a body part, i.e. a hand, a foot, etc., of a user, for example.

The support region may be integral or formed in one piece with the support body. In this variant, the support region typically forms an (exposed) surface of the support body. An (exposed) surface of the support body therefore has an individually configured geometric-structural design at least in portions, optionally completely.

It is, however, also conceivable for the support region to be designed as a component that is separate from the support body and, when required, is or can be connected, optionally in a detachable manner (without being damaged or destroyed), to the support body so as to form the support device. This variant may have advantages in terms of manufacturing, since only the support region, designed here as a component that is separate from the support body, has to be produced with an individually configured geometric-structural design. The support body can, however, be produced with a standardised design.

In this last-mentioned variant, the support body and/or the support region may comprise suitable, optionally corresponding, connection elements (not shown), which allow for a stable, but nevertheless typically detachable (without being damaged or destroyed) connection of the support body to the support region, and vice versa. The support body may e.g. comprise at least one first connection element and the support region may comprise at least one second connection element, the at least one first and the at least one second connection element being configured to interact with one another so as to form the support device. The at least one first connection element and the at least one second connection element may e.g. be configured to interact with one another in a form-fitting and/or force-locked manner so as to form the support device. It is also conceivable for the at least one first connection element and the at least one second connection element to be configured to interact with one another magnetically so as to form the support device.

In embodiments, the support region may comprise one or more individually configured support-region portions, which are in particular designed as receiving portions and are configured to receive a particular portion of a body part, in particular a limb portion, for example a finger or a toe, of a body part that is supported or is to be supported on the support region, i.e. in particular a limb. A support device intended to support a particular body part may therefore comprise a number of support-region portions that are individually configured specifically to this body part. Respective individually configured support-region portions may e.g. be designed as individually configured receiving portions. A relevant individually configured receiving portion may be designed for receiving one or more body parts. Typically, a relevant individually configured receiving portion is designed for receiving (exactly) one particular body part. The support region may comprise a number and/or arrangement of corresponding individually configured support-region portions which corresponds to the number and/or arrangement of the limb portions, i.e. the fingers or toes, for example, of a relevant body part to be supported on the support region. Using the example of a hand to be supported on the support region, the support region can therefore comprise five support-region portions, with each support-region portion supporting a particular finger of the hand to be supported. The same applies to other body parts.

The individually configured geometric-structural design, i.e. in particular geometric-structural parameters such as length, height, width, etc., of a relevant receiving portion is therefore adapted to or modelled on the anatomical form or morphology of the relevant body part to be received in the relevant receiving portion; the individually configured geometric-structural design of the receiving portion is therefore produced in respect of the morphology of a body part to be received therein. A relevant individually configured receiving portion may e.g. be designed as or comprise at least one depression e.g. formed by a recess or may e.g. be designed as or comprise at least one raised portion e.g. formed by a protrusion.

Using the example of a support device intended for supporting a hand, one or more individually configured receiving portions can therefore be provided for one or more fingers and/or one or more individually configured receiving portions can be provided for the palm. Using the example of a support device intended for supporting a foot, this typically not being the sole of a shoe, one or more individually configured receiving portions can therefore be provided for one or more toes and/or one or more individually configured receiving portions can therefore be provided for the sole of the foot. The result of the support and/or the relaxation effect on the relevant body part that can be achieved thereby can be influenced in a targeted manner by the type, number and level of customisation of respective receiving portions.

In embodiments, the support device may comprise a detection device, in particular integrated in the support region and/or the support body, which is configured to detect at least one chemical and/or physical variable of the support region and/or the support body and/or a limb supported on the support region. A corresponding detection device may e.g. be configured to detect chemical parameters, such as the composition of the surface of a supported body part, and/or thermal parameters, such as the temperature, and/or mechanical parameters, such as forces exerted on the support region and/or the support body, in particular by means of a supported body part, i.e. in particular weight forces, pressure, stress, etc., and/or hydrometric parameters, such as moisture, etc. A corresponding detection device may comprise one or more detection elements that are e.g. designed as or comprise sensors, which elements are arranged or formed on or in the support body and/or on or in the support region. The specific design of respective detection elements is determined by the chemical and/or physical variables to be detected in each case. A corresponding detection device typically has suitable hardware-implemented and/or software-implemented evaluation and/or processing measures, i.e. suitable evaluation and/or processing algorithms, for example, which allow for data-related evaluation and/or processing of respective detection signals supplied by the detection elements.

In embodiments, the support device may further comprise a transmission device, which is or can be assigned to the detection device, in particular is integrated in the support region and/or support body, and is configured to transmit a piece of detection information describing a chemical and/or physical variable detected by means of the detection device to at least one communication partner. A corresponding transmission device may transmit corresponding detection information to at least one communication partner in a wired or wireless manner. Wireless transmission may e.g. take place by means of a wireless connection or a wireless network via known industry standards, such as Bluetooth, for the data transmission. In this process, corresponding detection information may be transmitted in an unencrypted or encrypted manner. A communication partner may e.g. be a mobile terminal, such as a portable computer (laptop), a smartphone, a tablet, or smart glasses, may be a stationary terminal, such as a stationary computer (desktop, server), may be a network application, or may be network storage (cloud), etc.

In embodiments, the support device may further comprise a detection device, in particular integrated in the support region and/or the support body, which is configured to detect a body part supported on the support region. A corresponding detection device may e.g. be configured to detect when a body part supported or to be supported on the support region approaches or contacts the support region. A corresponding detection device may comprise one or more detection elements that are e.g. designed as or comprise sensors, in particular proximity sensors or contact sensors, which elements are arranged or formed on or in the support body and/or on or in the support region. A corresponding detection device typically has suitable hardware-implemented and/or software-implemented evaluation and/or processing measures, i.e. suitable evaluation and/or processing algorithms, for example, which allow for data-related evaluation and/or processing of respective detection signals supplied by the detection elements. A corresponding detection device may, however, also be configured to be identical to the above-mentioned detection device for detecting a chemical and/or physical variable, since conclusions can be drawn on a body part supported on the support region on the basis of respective detected chemical and/or physical variables.

In embodiments, the support device may further comprise a transmission device, which is or can be assigned to the detection device, in particular is integrated in the support region and/or support body, and is configured to transmit a piece of detection information describing a body part supported on the support region detected by means of the detection device to at least one communication partner. A corresponding transmission device may transmit corresponding detection information to at least one communication partner in a wired or wireless manner. Wireless transmission may e.g. take place by means of a wireless connection or a wireless network via known industry standards, such as Bluetooth, for the data transmission. In this process, corresponding detection information may be transmitted in an unencrypted or encrypted manner. A communication partner may e.g. be a mobile terminal, such as a portable computer (laptop), a smartphone, a tablet, or smart glasses, may be a stationary terminal, such as a stationary computer (desktop, server), may be a network application, or may be network storage (cloud), etc.

In embodiments, the support device may further comprise a detection device, in particular integrated in the support region and/or the support body, which is configured to detect at least one physiological variable, in particular the blood pressure, oxygen saturation or pulse, of a user supporting a body part on the support region. A corresponding detection device may comprise one or more detection elements that are e.g. designed as or comprise sensors, in particular blood-pressure, oxygen-saturation or pulse sensors, which elements are arranged or formed on or in the support body and/or on or in the support region. Corresponding physiological variables may e.g. be detected via the contact region between the support region and the relevant body part supported on the support region. A corresponding detection device typically has suitable hardware-implemented and/or software-implemented evaluation and/or processing measures, i.e. suitable evaluation and/or processing algorithms, for example, which allow for data-related evaluation and/or processing of respective detection signals supplied by the detection elements.

In embodiments, the support device may further comprise a transmission device, which is or can be assigned to the detection device, in particular is integrated in the support region and/or support body, and is configured to transmit a piece of detection information describing a physiological variable detected by means of the detection device to at least one communication partner. A corresponding transmission device may transmit corresponding detection information to at least one communication partner in a wired or wireless manner. Wireless transmission may e.g. take place by means of a wireless connection or a wireless network via known industry standards, such as Bluetooth, for the data transmission. In this process, corresponding detection information may be transmitted in an unencrypted or encrypted manner. A communication partner may e.g. be a mobile terminal, such as a portable computer (laptop), a smartphone, a tablet, or smart glasses, may be a stationary terminal, such as a stationary computer (desktop, server), may be a network application, or may be network storage (cloud), etc.

In embodiments, the support device may further comprise a detection device which is configured to detect a movement of the support body in at least one degree of freedom, in particular relative to a surface, and/or to detect a movement of a body part supported on the support region in at least one degree of freedom, in particular relative to the support body. A corresponding detection device may comprise one or more detection elements that are e.g. designed as or comprise sensors, in particular motion sensors, which elements are arranged or formed on or in the support body and/or on or in the support region. A corresponding detection device typically has suitable hardware-implemented and/or software-implemented evaluation and/or processing measures, i.e. suitable evaluation and/or processing algorithms, for example, which allow for data-related evaluation and/or processing of respective detection signals supplied by the detection elements.

In embodiments, the support device may further comprise a transmission device, which is or can be assigned to the detection device, in particular is integrated in the support region and/or support body, and is configured to transmit a movement, detected by means of the detection apparatus, of the support body in at least one degree of freedom, in particular relative to a surface, and/or to transmit a movement, detected by means of the detection apparatus, of a limb supported on the support body in at least one degree of freedom, in particular relative to the support body, to at least one communication partner. A corresponding transmission device may transmit corresponding detection information to at least one communication partner in a wired or wireless manner. Wireless transmission may e.g. take place by means of a wireless connection or a wireless network via known industry standards, such as Bluetooth, for the data transmission. In this process, corresponding detection information may be transmitted in an unencrypted or encrypted manner. A communication partner may e.g. be a mobile terminal, such as a portable computer (laptop), a smartphone, a tablet, or smart glasses, may be a stationary terminal, such as a stationary computer (desktop, server), may be a network application, or may be network storage (cloud), etc.

In embodiments, the support device may further comprise a storage device, in particular integrated in the support region and/or the support body, which is configured to store at least one piece of detection information detected by means of the detection device. A corresponding storage device is typically designed as or comprises at least one data storage means. A corresponding storage device is typically assigned a data-transmission interface, by means of which data can be read from the storage device in a wired or wireless manner and/or by means of which data can be input into the storage device.

In embodiments, the support device may further comprise a signal-output device, in particular integrated in the support region and/or the support body, which is configured to generate at least one in particular acoustic and/or optical and/or haptic output signal, in particular on the basis of at least one piece of detection information generated by the or a detection device. Certain output signals, generally signals, can therefore be output via a corresponding signal-output device by means of the support device. For example, an output signal indicating that a body part is properly supported on the support region can be output. A corresponding output signal may be generated or have been generated for example on the basis of detection information supplied by at least one detection device, i.e. a piece of detection information describing that a body part has been detected as contacting the support region, for example. A corresponding signal-output device typically comprises at least one signal-output element, which, depending on the specific signal to be output, may e.g. be designed as or comprise at least one acoustic signal-output element, i.e. a speaker, for example, as an optical signal-output element, i.e. a display element, light-emitting diode element, etc., for example, or as a haptic signal-output element, i.e. a vibration element, for example.

An output signal which can be output by means of a signal-output device, by way of example, may contain at least one piece of information relating to a user-side action and/or an evaluation of a user-side action, in particular on a user, of the support device. In this way, it is possible to assist a user in properly using the support device by means of corresponding output signals containing corresponding information. For example, it can be output to a user by corresponding output signals that the support device is ready to support a body part, and optionally also which body part, that a body part is properly supported or is not (yet) properly supported, or when/that a particular support duration, e.g. expedient for a therapeutic effect, has been reached or has not (yet) been reached, etc. Equally, output signals that encourage or motivate a user are conceivable, i.e. output signals that encourage or motivate a user, e.g. acoustically, optically or haptically, to use the support device, to assume and/or maintain a particular support position, etc. Similarly, output signals that assess, i.e. in particular praise or criticise, a user are conceivable, which signals assess a user or a particular user action, e.g. acoustically, optically or haptically, before and/or during and/or after use of the support device.

In embodiments, the support body may be integrated in an input apparatus of a device, i.e. a terminal, for example, such as a computer, a multimedia apparatus, etc., that can be controlled by an input apparatus, or is designed as an input apparatus of this kind. In this way, increased functional integration can be achieved, which also gives the user the option of carrying out particular actions, i.e. in particular inputs, controls, etc., using the body part while the body part is being supported. A specific example of a corresponding input apparatus is an input element, e.g. designed as an (electronic) computer mouse, for making inputs into a computer, an input element, e.g. designed as a remote control, for making inputs into a multimedia apparatus, such as a television, an audio system, etc. Other examples are conceivable.

In embodiments, the support device may comprise a temperature-control device, in particular integrated in the support region and/or the support body, for controlling the temperature of the support region and/or the support body at least in portions, optionally completely. The static or dynamic temperature control (understood in principle as both heating and cooling) of the support region and/or the support body, made possible by means of a corresponding temperature-control device, can assist the desired relaxation of a relevant body part that is being supported or can assist a therapeutic effect that can be achieved by a relevant body part being supported.

A corresponding temperature-control device may comprise a temperature-control duct structure comprising a temperature-control duct through which a temperature-control medium, i.e. a gas or liquid, for example, can flow. A corresponding temperature-control duct structure may extend through the support body or the support region, in particular in a planar manner, at least in portions, optionally completely. In particular, a corresponding temperature-control duct structure may extend, in particular with close contours, in the region of or below the (exposed) surface of the support body or the support region forming an actual support surface of the support body or the support region.

Alternatively or additionally, a corresponding temperature-control device may comprise at least one electrically operated temperature-control element. A corresponding electrically operated temperature-control element may e.g. be designed as or comprise at least one heating wire or a one-dimensional or multi-dimensional heating-wire structure. A corresponding temperature-control element may extend through the support body or the support region, in particular in a planar manner, at least in portions, optionally completely. In particular, a corresponding temperature-control element may extend, in particular with close contours, in the region of or below the (exposed) surface of the support body or the support region forming an actual support surface of the support body or the support region.

Alternatively or additionally, a corresponding temperature-control device may comprise at least one temperature-control element, which performs an optionally reversible exothermic chemical reaction after activation. A corresponding temperature-control element may be arranged or formed in the region of or below the (exposed) surface of the support body or the support region forming an actual support surface of the support body or the support region. A corresponding temperature-control element may comprise an activation element, i.e. a tab, a button, etc., by means of which it is possible to activate the temperature-control element, i.e. to initiate the exothermic reaction.

In embodiments, the support device may further comprise a fixing device, which is or can be assigned to the support body and/or the support region, for temporarily fixing a body part supported on the support region, in particular in an intended support position of the body part on the support region.

Corresponding fixing may facilitate and maintain a desired orientation of a body part supported on the support region. In this way, a relaxation effect or therapeutic effect that can be produced by the support device can be assisted. A corresponding fixing device can make it possible to fix a relevant body part directly to the support body or the support region; this may e.g. be implemented by a fixing element that is or can be attached to the support body or the support region, such as a strap, a loop, etc., which surrounds the relevant body part at least in portions, optionally completely, after being put on, such that the body part is fixed to the support body or the support region. A tube-like or tubular fixing device is also conceivable, i.e. a fixing tube made of a fabric, for example, which can be pulled over the support device and the body part that is supported or is to be supported thereon and, in this way, a desired orientation of a body part supported on the support region is facilitated and maintained. A fixing device is also conceivable which makes it possible to fix a superordinate body part, i.e. an arm, for example, in a particular orientation when the body part to be fixed is a hand.

In embodiments, the support body may comprise at least one mounting portion for mounting the support body on a surface or a base. Depending on the design, the support body can be mounted in a stable manner on a surface or a base, such as a table surface, by means of a corresponding mounting portion.

A corresponding mounting portion may be provided with or comprise at least one material or corresponding material structure that provides non-slip support of the support body on a surface or a base. For example, a corresponding material or a corresponding material structure may therefore be expedient for allowing the support body to be supported in a stable manner on a surface or a base. A corresponding material or a corresponding material structure may e.g. be made of an elastomer material or an elastomer material structure or may comprise at least one elastomer material. In principle, both natural and synthetic elastomer materials or material structures are possible. In terms of manufacturing, thermoplastic elastomers may advantageously be used, for example.

A corresponding mounting portion may be provided with or comprise at least one reversibly deformable material, in particular a reversibly deformable, flexible material, or a reversibly deformable material structure, in particular a reversibly deformable, flexible material structure. For example, a corresponding material or a corresponding material structure may therefore be expedient to allow any required modifications to the orientation of the support body on a surface or a base, in particular relative to a neutral or reference orientation, which, for example, can make it easier or make it possible for users with spastic body parts or limbs to use the support device; this is particularly applicable if the spasticity in question or another anatomical malalignment requires a particular orientation of the support body, in particular relative to a neutral or reference orientation, in order to support the body part on the support region, in particular without causing any pain. A corresponding material or a corresponding material structure may e.g. be made of an elastomer or cellular material or an elastomer or cellular material structure or may comprise at least one elastomer material. In principle, both natural and synthetic elastomer or cellular materials or material structures are possible. In terms of manufacturing, optionally foamed, thermoplastic elastomers may advantageously be used, for example.

A corresponding mounting portion may comprise at least one mounting-portion element which in particular lies in a non-planar manner on a planar surface or base and makes it possible to mount the support body that is mounted on the surface relative to the surface or base so as to be movable, in particular pivotally movable, in at least one degree of freedom. In this way, the support body can be moved relative to a surface or a base, which may be advantageous for producing the most relaxed possible position of a relevant body part and superordinate body parts. For example, an orientation of the support body that can be modified compared with a reference orientation allows for (yet) more relaxing or relaxed support of a body part. A corresponding mounting-portion element may e.g. have a monogonal, polygonal, circular, round or pointed geometry. Likewise, movably mounting the support body can provide training options, e.g. for building muscle, since by moving the support body relative to the surface or the base, certain movement sequences associated with a training effect can be performed.

A corresponding mounting-portion element may also be designed as a separate component and, when required, is or can be connected, optionally in a detachable manner (without being damaged or destroyed), to the support body so as to form the support device. A corresponding, e.g. spherical, hemispherical, cylindrical or semi-cylindrical or pyramidal mounting-portion element may e.g. be connected to the support body by a hook-and-loop fastener system or an interchangeable fastener system.

In embodiments, the support device may comprise at least one support element, which is or can be supported on the support region, in particular detachably (without being damaged or destroyed), so as to cover said support region at least in portions, optionally completely. A corresponding support element may in particular be expedient from a hygiene viewpoint, since the support region or the support body is protected against direct contact with the relevant body part and thus e.g. against deposits of skin, sweat, etc. A corresponding support element may be made of a resiliently flexible material or a resiliently flexible material structure, such that the support element does not impair the fit of the support region. A corresponding support element may be made of an optionally washable, natural and/or synthetic fabric material or a natural and/or synthetic fabric material structure. Merely by way of example, reference is made to leather or plastics materials.

In embodiments, the support region and/or the support body may be made of or comprise at least one magnetisable or magnetic material. Magnetisable or magnetic materials, such as corresponding ferromagnetic metals, may have a positive effect on the blood flow through the corresponding body part, and this can promote therapeutic effects. Magnetisable or magnetic materials may be provided as a solid material or in the form of particles.

Alternatively or additionally, the support region and/or the support body may be made of or comprise at least one antibacterial material. Antibacterial materials, such as antibacterial metals, may have a positive effect on the hygienic properties of the support region and/or the support body. Antibacterial materials may be provided as a solid material or in the form of particles.

With regard to the production of the support device, i.e. the support body and/or the support region, in principle any manufacturing methods, in particular moulding and/or shaping manufacturing methods, are taken into consideration.

In particular, both additive (build-up) manufacturing methods and subtractive (material-removing) manufacturing methods, i.e. in particular machining manufacturing methods, are taken into consideration.

Additive manufacturing methods may be particularly expedient, since these are particularly suitable for producing corresponding, individually configured geometric-structural designs. Therefore, additive manufacturing methods can be taken into consideration which allow for the additive build-up of three-dimensional objects by selectively hardening, in layers, an optionally powdered construction material that can be selectively hardened in layers (this may e.g. be a metal or a plastics material) by means of radiation energy, i.e. in particular by means of at least one energy beam. Merely by way of example, in this connection reference is made to binder-jetting methods, selective laser-melting methods, selective laser-sintering methods or sterolithography methods.

In addition to the support device, embodiments described herein also relate to a communication system which comprises at least one support device as described herein, which comprises a transmission device, which is configured to transmit a piece of information to at least one communication partner and/or to receive a piece of information transmitted by at least one communication partner, and at least one communication partner, which is configured to receive a piece of information transmitted by the at least one support device and/or to transmit a piece of information to the at least one support device.

All the embodiments relating to the support device, in particular all the embodiments relating to any transmission devices of the support device, apply by analogy to the communication system.

Moreover, the present specification relates to a method for producing a support device for supporting at least one body part of a user at least in portions, optionally completely, comprising a support body, which comprises at least one support region for supporting at least one body part of a user at least in portions, optionally completely. The method comprises the steps of:

providing data describing at least one body part of a user that is to be supported on the support region of the support device to be produced at least in portions, optionally completely,

producing at least the support region on the basis of the data such that the support region has, at least in portions, optionally completely, a geometric-structural design that is individually configured on the basis of the data.

All the embodiments relating to the support device, in particular embodiments relating to manufacturing-related aspects of the support device, apply by analogy to the method.

The method may further contain a step of recording data describing at least one body part of a user that is to be supported on the support region at least in portions, optionally completely. Recording corresponding data that corresponds to the subsequent provision may therefore be part of the method.

Corresponding data may e.g. be recorded by means of a raw-data recording device, which is e.g. designed as or comprises at least one in particular mechanical or optical recording device which is configured to record one-dimensional or multi-dimensional body-part raw data describing the morphology of at least one body part. A mechanical recording device may e.g. be designed as a pressure-measuring device, which makes it possible to record body-part raw data describing the morphology of at least one body part on the basis of recorded mechanical variables, i.e. in particular pressure. A mechanical recording device may in particular also be configured to create a body-part impression. An optical recording device may e.g. be designed as a camera device or a scanning device, in particular a body-part scanning device. In any case, it is also conceivable for a corresponding raw-data recording device to be designed as or comprise at least one recording device or detection device for in particular mechanically and/or optically recording or detecting one-dimensional or multi-dimensional dimensions, i.e. the length, width, height, periphery or volume, of a body part.

The support body and/or the support region may, as already mentioned in connection with the support device, in particular be produced by means of an additive manufacturing method. Nevertheless, other manufacturing methods are conceivable.

### BRIEF DESCRIPTION OF THE DRAWINGS

The support devices described herein are explained in greater detail on the basis of embodiments in the drawings, in which:

FIG. 1a is a perspective front view of a support device for supporting a body part of a user according to an embodiment;

FIG. 1b is a perspective rear view of a support device for supporting the body part of a user of FIG. 1a according to an embodiment;

FIG. 1c is a sectional view of a support device for supporting the body part of a user of FIG. 1a according to an embodiment;

FIG. 2 is a schematic view of a support device for supporting a body part of a user according to an embodiment;

FIG. 3 is a schematic view of a support device for supporting a body part of a user according to an embodiment;

FIG. 4 is a schematic view of a support device for supporting a body part of a user according to an embodiment;

FIG. 5 is a schematic view of a support device for supporting a body part of a user according to an embodiment;

FIG. 6 is a schematic view of a support device for supporting a body part of a user according to an embodiment;

FIG. 7 is a schematic view of a support device for supporting a body part of a user according to an embodiment;

FIG. 8 is a schematic view of a support device for supporting a body part of a user according to an embodiment;

FIG. 9 is a schematic view of a support device for supporting a body part of a user according to an embodiment;

FIG. 10 is a schematic view of a communication system according to an embodiment; and

FIG. 11 is a schematic view of a method for producing a support device for receiving a body part of a user according to an embodiment.

### DETAILED DESCRIPTION

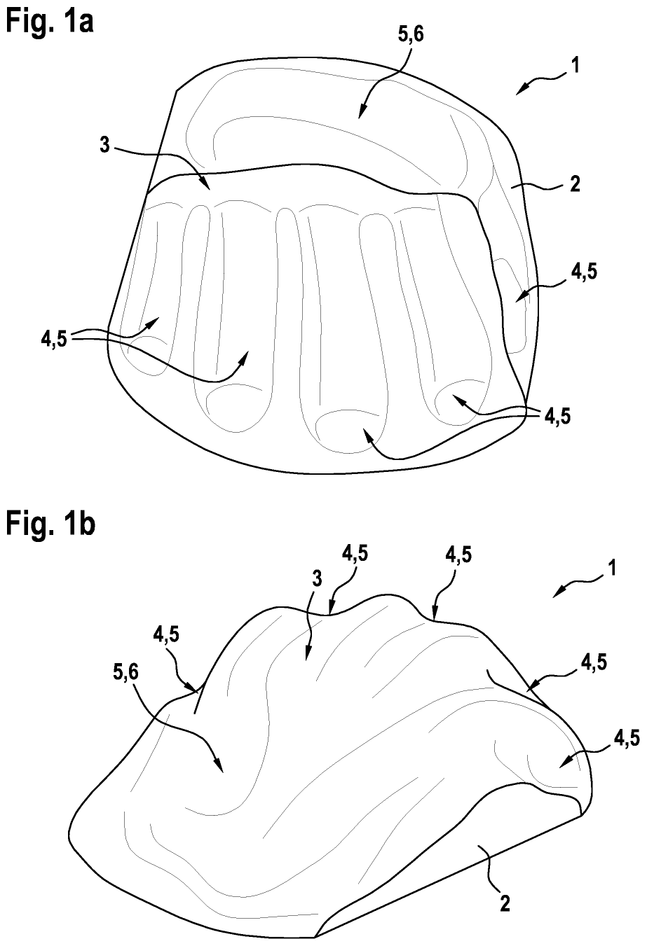

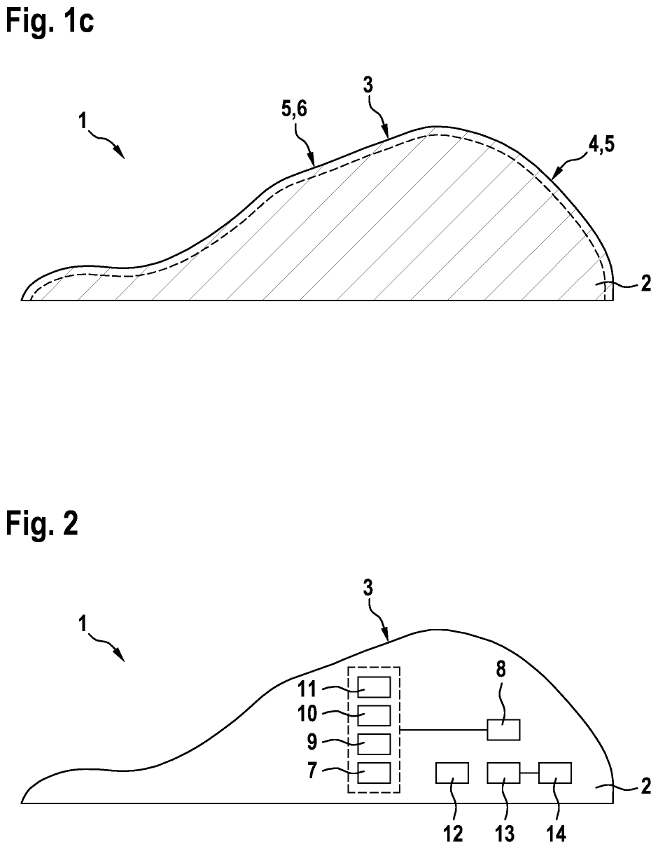

FIGS. 1a-1c depict a support device 1 for supporting a body part (not shown) of a user according to an embodiment. In general, the support device 1 serves to support or mount at least one body part, i.e. a hand of a user, for example. FIG. 1a is a perspective front view of the support device 1, FIG. 1b is a perspective rear view of said support device, and FIG. 1c is a sectional view of said support device along the lines ii-ii shown in FIG. 1b.

The support device 1 comprises a support body 2. The support body 2, which can also be referred to as or considered to be a mounting body, also comprises a support region 3, which can also be referred to as or considered to be a mounting region, for supporting a body part of a user. The support region 3 is therefore understood to be the region of the support device 1 on which a user (directly) supports a body part when the support device 1 is used as intended. The support region 3 therefore constitutes the actual region of the support device 1 on which a user supports or mounts a body part when the support device 1 is used as intended. As shown in FIG. 1, the support region 3 typically forms at least one part of the (exposed) surface of the support device 1.

As shown in FIG. 1, the support device 1 therefore substantially consists of a block-like or block-shaped support body 2, which comprises a surface which has three-dimensional contours or structures that are individually configured in respect of a particular body part of a particular user. These three-dimensional contours or structures are formed by the support region 3 in this case.

The dimensions of the support device 1, the support body 2 and the support region 3 are typically adapted to the dimensions of the body part to be supported in each case. When the support device 1 is used as intended, the support region 3 can be completely covered by the body part being supported in each case.

The support device 1 is characterised in that the support region 3 has a geometric-structural design that is individually configured on the basis of the data describing the body part of a user that is to be supported on the support region 3. The support region 3 therefore has an individually configured geometric-structural design and thus has a high degree of customisation. The support region 3, and optionally also the support body 2 and thus also the support device 1, may therefore be a component that is individually configured or individually configurable for a particular body part of a particular user.

The individually configured geometric-structural design of the support region 3, with this in particular being understood to be particular contours or structures of the surface of the support region 3, has been produced on the basis of the data describing at least one body part of a particular user that is to be supported on the support region 3 when used as intended. The support region 3 therefore typically constitutes a (negative) reproduction, individually configured in a user-specific manner, of a body part of a particular user that is to be supported on the support region 3. This allows for a fit of the support region 3 that is individually configured in a user-specific manner or a manner specific to a body part of the user and is thus highly precise, this fit allowing the relevant body part to be supported on the support region 3 in a relaxing or relaxed position specific to this body part.

Supporting a body part in a relaxing or relaxed position specific to this body part can both have a relaxing effect on the relevant body part as well as on other body parts adjacent to this body part, such that the user's whole body can be brought into a relaxing or relaxed state when using the support device 1. In addition to the mere relaxing effect or as a consequence thereof, where necessary, therapeutic effects can also be brought about, since supporting the body part in a relaxing or relaxed position specific to this body part can e.g. have a therapeutic effect on the blood flow and/or oxygen supply to the relevant body part. Likewise, the effect of reducing swelling could also be demonstrated. Positive effects could be identified in particular in rheumatic body parts or limbs and/or in e.g. spastic body parts or limbs due to a stroke. The increase in the internal tension (tone) of the muscles of the relevant body part, associated with spasticity, could be reduced (significantly in some cases) by (regular) use of the support device 1.

The user can use the support device 1 in particular without any help from a third party; therefore, the user can independently carry out therapeutic measures, i.e. for regulating tone, pain therapy, etc., and prepare themselves for corresponding therapeutic measures in the sense of self-help by using the support device 1 as intended.

Using the example of a spastic hand, the support device 1, which, in this respect, can be referred to as or considered to be a customised support for the heel of the hand for providing therapy to persons with a spastic or cramped hand, makes it possible for the hand to rest on the support or support device 1 in a loosened state. By supporting the cramped hand on the customised support surface, a state of relaxation is achieved. The hand is reproduced and positioned by the shape of the customised support for the heel of the hand with a precise fit.

With reference to FIG. 1, it is clear that the support region 3 may be integral or formed in one piece with the support body 2. The support region 3 forms an (exposed) surface of the support body 2 here. An (exposed) surface of the support body 2 therefore has an individually configured geometric-structural design.

On the basis of FIG. 1c, as shown by the dashed line, it is, however, also conceivable for the support region 3 to be designed as a component that is separate from the support body 2 and, when required, is or can be connected, optionally in a detachable manner (without being damaged or destroyed), to the support body 2 so as to form the support device 1. This variant may have advantages in terms of manufacturing, since only the support region 3, designed here as a component that is separate from the support body 2, has to be produced with an individually configured geometric-structural design. The support body 2 can, however, be produced with a standardised design.

In this last-mentioned variant, the support body 2 and/or the support region 3 may comprise suitable, optionally corresponding, connection elements (not shown), which allow for a stable, but nevertheless typically detachable (without being damaged or destroyed) connection of the support body 2 to the support region 3, and vice versa. The support body 2 may e.g. comprise at least one first connection element and the support region 3 may comprise at least one second connection element, the at least one first and the at least one second connection element being configured to interact with one another so as to form the support device 1. The at least one first connection element and the at least one second connection element may e.g. be configured to interact with one another in a form-fitting and/or force-locked manner so as to form the support device 1. It is also conceivable for the at least one first connection element and the at least one second connection element to be configured to interact with one another magnetically so as to form the support device 1.

With reference to FIG. 1, it is clear that the support region 3 may comprise a plurality of individually configured support-region portions 5, which are in particular designed as receiving portions 4 and are configured to receive a particular body part, in particular a limb portion, of a body part, i.e. in particular a limb, that is supported or is to be supported on the support region 3. The support device 1 intended to support a particular body part may therefore comprise a number of support-region portions 5 that are individually configured specifically to this body part. Respective individually configured support-region portions 5 may e.g. be designed as individually configured receiving portions 4. As shown in FIG. 1, a relevant receiving portion 4 is typically designed to receive (exactly) one particular body part, i.e. a finger in the embodiment. It is clear that the support region 3 may comprise a number and/or arrangement of corresponding individually configured support-region portions 5 which corresponds to the number and/or arrangement of the limb portions, i.e. the fingers or toes, for example, of a body part to be supported on the support region 3.

In the embodiment, shown in FIG. 1, of a hand to be supported on the support region 3, the support region 3 therefore comprises five support-region portions 5, with each support-region portion 5 supporting a particular finger of the hand to be supported. The same applies to other body parts.

Specifically, the embodiment according to FIG. 1 shows corresponding support-region portions 5 or receiving portions 4 for each finger on a hand; FIG. 1a shows, on the left, a corresponding support-region portion 5 for the little finger, beside a corresponding support-region portion 5 for the ring finger, beside a corresponding support-region portion 5 for the middle finger, beside a corresponding support-region portion 5 for the index finger, beside, i.e. on the far right, a corresponding support-region portion 5 for the thumb.

The individually configured geometric-structural design, i.e. in particular geometric-structural parameters such as length, height, width, etc., of relevant receiving portions 4 is therefore adapted to or modelled on the anatomical form or morphology of the relevant body part to be received in the relevant receiving portion 4; the individually configured geometric-structural design of the relevant receiving portion 4 is therefore produced in respect of the morphology of a body part to be received therein. With reference to FIG. 1, it is clear that a relevant receiving portion 4 may e.g. be designed as or comprise at least one depression e.g. formed by a recess. It is, however, equally conceivable for a relevant receiving portion 4 to e.g. be designed as or comprise at least one raised portion e.g. formed by a protrusion.

In the embodiment, shown in FIG. 1, of a support device 1 intended for supporting a hand, a plurality of individually configured receiving portions 4 are clearly provided for one or more fingers and an individually configured receiving portion 6 is provided for the palm. Using the example of a support device 1 intended for supporting a foot, this typically not being the sole of a shoe, one or more individually configured receiving portions could accordingly be provided for one or more toes and/or one or more individually configured receiving portions could accordingly be provided for the sole of the foot. The result of the support and/or the relaxation effect on the relevant body part that can be achieved thereby can be influenced in a targeted manner by the type, number and level of customisation of respective receiving portions.

FIG. 10 is a schematic side view of a support device 1 for supporting a body part (not shown) of a user according to an embodiment.

With reference to the embodiment shown in FIG. 2, it is clear that the support device 1 comprises a detection device 7, in particular integrated in the support body 2, which is configured to detect at least one chemical and/or physical variable of the support region 3 and/or the support body 2 and/or a limb supported on the support region 3. A corresponding detection device 7 may e.g. be configured to detect chemical parameters, such as the composition of the surface of a supported body part, and/or thermal parameters, such as the temperature, and/or mechanical parameters, such as forces exerted on the support region 3 and/or the support body 2, in particular by means of a supported body part, i.e. in particular weight forces, pressure, stress, etc., and/or hydrometric parameters, such as moisture, etc. A corresponding detection device 7 may comprise one or more detection elements (not shown) that are e.g. designed as or comprise sensors, which elements are arranged or formed on or in the support body 2 and/or on or in the support region 3. The specific design of respective detection elements is determined by the chemical and/or physical variables to be detected in each case. A corresponding detection device 7 typically has suitable hardware-implemented and/or software-implemented evaluation and/or processing measures, i.e. suitable evaluation and/or processing algorithms, for example, which allow for data-related evaluation and/or processing of respective detection signals supplied by the detection elements.

With reference to FIG. 2, it is also clear that the support device 1 may comprise a detection device 9, in particular integrated in the support body 2, which is configured to detect a body part supported on the support region 3. A corresponding detection device 9 may e.g. be configured to detect when a body part supported or to be supported on the support region 3 approaches or contacts the support region 3. A corresponding detection device 9 may comprise one or more detection elements (not shown) that are e.g. designed as or comprise sensors, in particular proximity sensors or contact sensors, which elements are arranged or formed on or in the support body 2 and/or on or in the support region 3. A corresponding detection device 9 typically has suitable hardware-implemented and/or software-implemented evaluation and/or processing measures, i.e. suitable evaluation and/or processing algorithms, for example, which allow for data-related evaluation and/or processing of respective detection signals supplied by the detection elements. A corresponding detection device 9 may, however, also be configured to be identical to the above-mentioned detection device 7 for detecting a chemical and/or physical variable, since conclusions can be drawn on a body part supported on the support region 3 on the basis of respective detected chemical and/or physical variables.

With reference to FIG. 2, it is also clear that the support device 1 may comprise a detection device 10, in particular integrated in the support body 2, which is configured to detect at least one physiological variable, in particular the blood pressure, oxygen saturation or pulse, of a user supporting a body part on the support region 3. A corresponding detection device 10 may comprise one or more detection elements (not shown) that are e.g. designed as or comprise sensors, in particular blood-pressure, oxygen-saturation or pulse sensors, which elements are arranged or formed on or in the support body 2 and/or on or in the support region 3. Corresponding physiological variables may e.g. be detected via the contact region between the support region 3 and the relevant body part supported on the support region 3. A corresponding detection device 10 typically has suitable hardware-implemented and/or software-implemented evaluation and/or processing measures, i.e. suitable evaluation and/or processing algorithms, for example, which allow for data-related evaluation and/or processing of respective detection signals supplied by the detection elements.

With reference to FIG. 2, it is also clear that the support device 1 may comprise a detection device 11, which is configured to detect a movement of the support body 2 in at least one degree of freedom, in particular relative to a surface, and/or to detect a movement of a body part supported on the support region 3 in at least one degree of freedom, in particular relative to the support body 2. A corresponding detection device 11 may comprise one or more detection elements (not shown) that are e.g. designed as or comprise sensors, in particular motion sensors, which elements are arranged or formed on or in the support body 2 and/or on or in the support region 3. A corresponding detection device 11 typically has suitable hardware-implemented and/or software-implemented evaluation and/or processing measures, i.e. suitable evaluation and/or processing algorithms, for example, which allow for data-related evaluation and/or processing of respective detection signals supplied by the detection elements.

As shown by the box formed by dashed lines, the detection devices 7, 9, 10, 11 may also be combined in a common detection device in terms of hardware and/or software.

With reference to FIG. 2, it is also clear that the support device 1 may comprise a transmission device 8, which is or can be assigned to the detection device(s) 7, 9, 10, 11, in particular is integrated in the support region 2 and/or support body 3, and is configured to transmit relevant detection information to at least one communication partner. A corresponding transmission device 8 may transmit corresponding detection information to at least one communication partner in a wired or wireless manner. Wireless transmission may e.g. take place by means of a wireless connection or a wireless network via known industry standards, such as Bluetooth, for the data transmission. In this process, corresponding detection information may be transmitted in an unencrypted or encrypted manner. A communication partner may e.g. be a mobile terminal, such as a portable computer (laptop), a smartphone, a tablet, or smart glasses, may be a stationary terminal, such as a stationary computer (desktop, server), may be a network application, or may be network storage (cloud), etc.

With reference to FIG. 2, it is also clear that the support device 1 may comprise a storage device 12, in particular integrated in the support region 2 and/or the support body 3, which is configured to store at least one piece of detection information detected by means of the detection device 7, 9, 10, 11. A corresponding storage device 12 is typically designed as or comprises at least one data storage means. A corresponding storage device 12 is typically assigned a data-transmission interface (not shown), by means of which data can be read from the storage device 12 in a wired or wireless manner and/or by means of which data can be input into the storage device 12.

With reference to FIG. 2, it is also clear that the support device 1 may comprise a signal-output device 13, in particular integrated in the support body 2, which is configured to generate at least one in particular acoustic and/or optical and/or haptic output signal, in particular on the basis of at least one piece of detection information generated by a detection device 7, 9, 10, 11. Certain output signals, generally signals, can therefore be output via a corresponding signal-output device 13 by means of the support device 1. For example, an output signal indicating that a body part is properly supported on the support region 3 can be output. A corresponding output signal may be generated or have been generated for example on the basis of detection information supplied by at least one detection device 7, 9, 10, 11, i.e. a piece of detection information describing that a body part has been detected as contacting the support region 3, for example. A corresponding signal-output device 13 typically comprises at least one signal-output element 14, which, depending on the specific signal to be output, may e.g. be designed as or comprise at least one acoustic signal-output element, i.e. a speaker, for example, as an optical signal-output element, i.e. a display element, light-emitting diode element, etc., for example, or as a haptic signal-output element, i.e. a vibration element, for example.

An output signal which can be output by means of a signal-output device 13, by way of example, may contain at least one piece of information relating to a user-side action and/or an evaluation of a user-side action, in particular on a user, of the support device 1. In this way, it is possible to assist a user in properly using the support device 1 by means of corresponding output signals containing corresponding information. For example, it can be output to a user by corresponding output signals that the support device 1 is ready to support a body part, and optionally also which body part, that a body part is properly supported or is not (yet) properly supported, or when/that a particular support duration, e.g. expedient for a therapeutic effect, has been reached or has not (yet) been reached, etc. Equally, output signals that encourage or motivate a user are conceivable, i.e. output signals that encourage or motivate a user, e.g. acoustically, optically or haptically, to use the support device 1, to assume and/or maintain a particular support position, etc. Similarly, output signals that assess, i.e. in particular praise or criticise, a user are conceivable, which signals assess a user or a particular user action, e.g. acoustically, optically or haptically, before and/or during and/or after use of the support device 1.

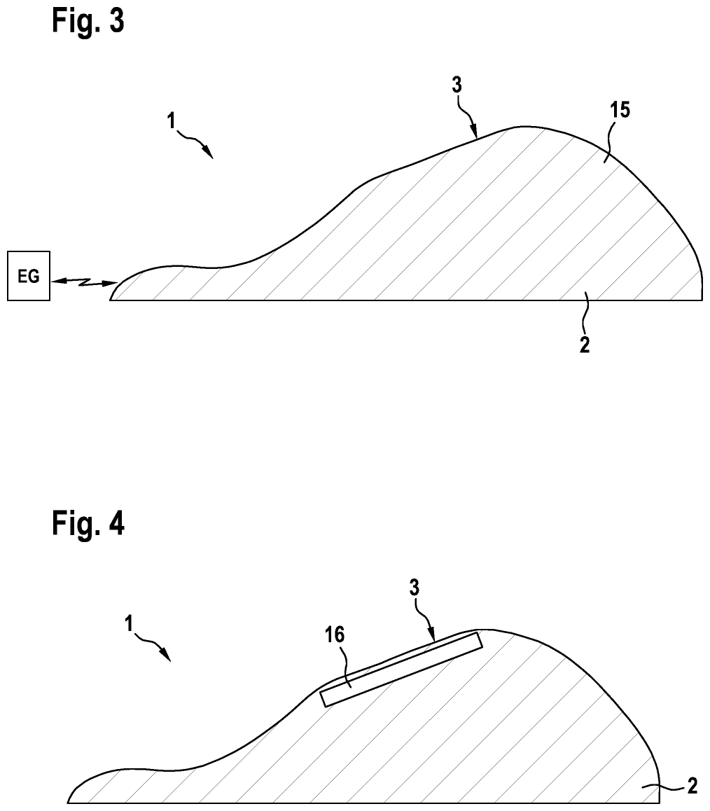

FIG. 3 is a schematic side view of a support device 1 for supporting a body part (not shown) of a user according to an embodiment.

With reference to FIG. 3, it is clear that the support body 2 may be integrated in an input apparatus 15 of a device (not shown), i.e. a terminal EG, for example, such as a computer, a multimedia apparatus, etc., that can be controlled by an input apparatus, or is designed as an input apparatus 15 of this kind. In this way, increased functional integration can be achieved, which also gives the user the option of carrying out particular actions, i.e. in particular inputs, controls, etc., using the body part while the body part is being supported. A specific example of a corresponding input apparatus 15 is, as shown in FIG. 2, an input element, e.g. designed as an (electronic) computer mouse, for making inputs, indicated by the arrow, into a computer, an input element, e.g. designed as a remote control, for making inputs into a multimedia apparatus, such as a television, an audio system, etc. Other examples are conceivable.

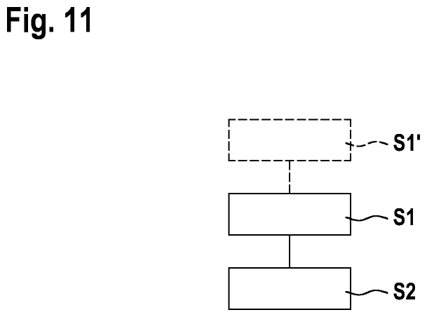

FIG. 11 is a schematic side view of a support device 1 for supporting a body part (not shown) of a user according to an embodiment.

With reference to FIG. 4, it is clear that the support device 1 may comprise a temperature-control device 16, in particular integrated in the support body 2, for controlling the temperature of the support region 3 and/or the support body 2 at least in portions, optionally completely. The static or dynamic temperature control (understood in principle as both heating and cooling) of the support region 3 and/or the support body 2, made possible by means of a corresponding temperature-control device 16, can assist the desired relaxation of a relevant body part that is being supported or can assist a therapeutic effect that can be achieved by a relevant body part being supported.

A corresponding temperature-control device 16 may comprise a temperature-control duct structure comprising a temperature-control duct through which a temperature-control medium, i.e. a gas or liquid, for example, can flow. A corresponding temperature-control duct structure may extend through the support body 2 or the support region 3, in particular in a planar manner, at least in portions, optionally completely. In particular, a corresponding temperature-control duct structure may extend, in particular with close contours, in the region of or below the (exposed) surface of the support body 2 or the support region 3 forming an actual support surface of the support body 2 or the support region 3.

Alternatively or additionally, a corresponding temperature-control device 16 may comprise at least one electrically operated temperature-control element (not shown). A corresponding electrically operated temperature-control element may e.g. be designed as or comprise at least one heating wire or a one-dimensional or multi-dimensional heating-wire structure. A corresponding temperature-control element may extend through the support body 2 or the support region 3, in particular in a planar manner, at least in portions, optionally completely. In particular, a corresponding temperature-control element may extend, in particular with close contours, in the region of or below the (exposed) surface of the support body 2 or the support region 3 forming an actual support surface of the support body 2 or the support region 3.

Alternatively or additionally, a corresponding temperature-control device 16 may comprise at least one temperature-control element, which performs an optionally reversible exothermic chemical reaction after activation. A corresponding temperature-control element may be arranged or formed in the region of or below the (exposed) surface of the support body 2 or the support region 3 forming an actual support surface of the support body 2 or the support region 3. A corresponding temperature-control element may comprise an activation element (not shown), i.e. a tab, a button, etc., by means of which it is possible to activate the temperature-control element, i.e. to initiate the exothermic reaction.

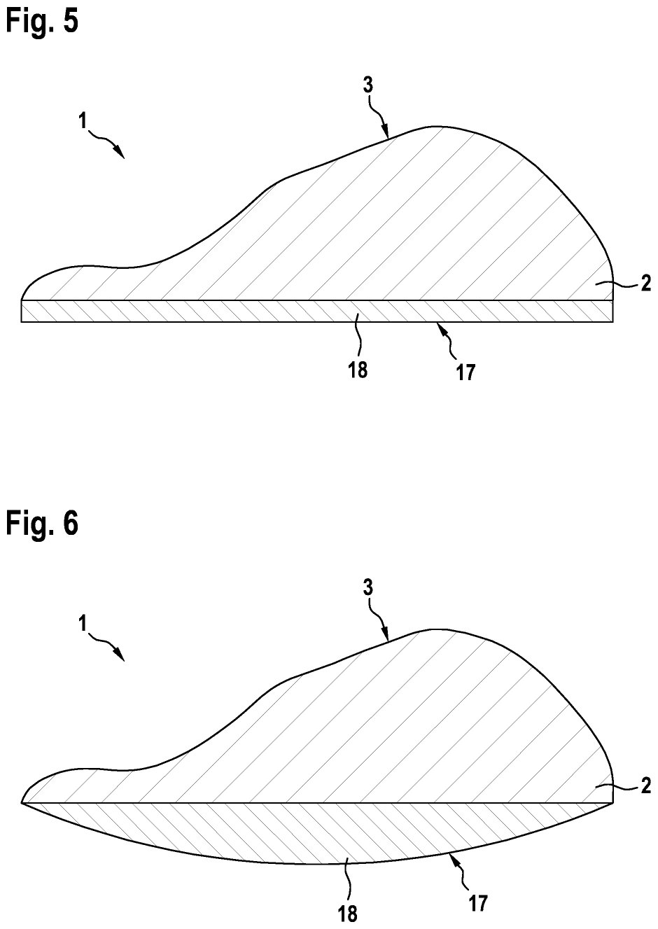

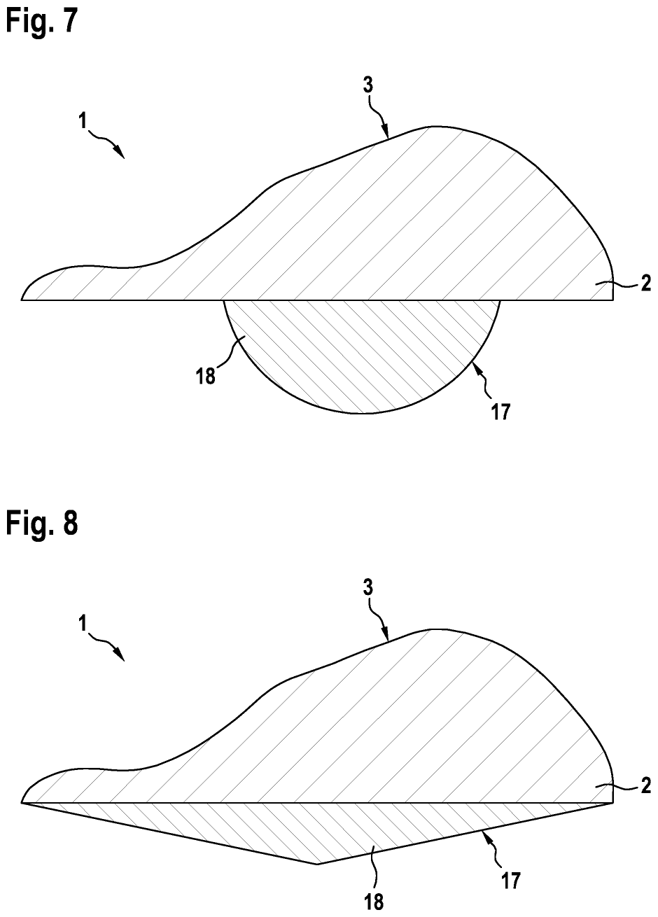

FIGS. 5-8 are each a schematic side view of a support device 1 for supporting a body part (not shown) of a user according to further embodiments.

With reference to FIGS. 5-8, it is clear that the support body 2 comprises a mounting portion 17 for mounting the support body 2 on a surface or a base. Depending on the design, the support body 2 can be mounted in a stable manner on a surface or a base, such as a table surface, by means of a corresponding mounting portion 17.

A corresponding mounting portion 17 may be provided with or comprise at least one material or corresponding material structure that provides non-slip support of the support body 2 on a surface or a base. For example, a corresponding material or a corresponding material structure may therefore be expedient for allowing the support body 2 to be supported in a stable manner on a surface or a base. A corresponding material or a corresponding material structure may e.g. be made of an elastomer material or an elastomer material structure or may comprise at least one elastomer material. In principle, both natural and synthetic elastomer materials or material structures are possible. In terms of manufacturing, thermoplastic elastomers may advantageously be used, for example.

A corresponding mounting portion 17 may be provided with or comprise at least one reversibly deformable material, in particular a reversibly deformable, flexible material, or a reversibly deformable material structure, in particular a reversibly deformable, flexible material structure. For example, a corresponding material or a corresponding material structure may therefore be expedient to allow any required modifications to the orientation of the support body 2 on a surface or a base, in particular relative to a neutral or reference orientation, which, for example, can make it easier or make it possible for users with spastic body parts or limbs to use the support device 1; this is particularly applicable if the spasticity in question or another anatomical malalignment requires a particular orientation of the support body 2, in particular relative to a neutral or reference orientation, in order to support the body part on the support region 3, in particular without causing any pain. A corresponding material or a corresponding material structure may e.g. be made of an elastomer or cellular material or an elastomer or cellular material structure or may comprise at least one elastomer material. In principle, both natural and synthetic elastomer or cellular materials or material structures are possible. In terms of manufacturing, optionally foamed, thermoplastic elastomers may advantageously be used, for example.

A corresponding mounting portion 17 may, as shown in FIGS. 6-8, comprise a mounting-portion element 18 which in particular lies in a non-planar manner on a planar surface or base and makes it possible to mount the support body 2 that is mounted on the surface relative to the surface or base so as to be movable, in particular pivotally movable, in at least one degree of freedom. In this way, the support body 2 can be moved relative to a surface or a base, which may be advantageous for producing the most relaxed possible position of a relevant body part and superordinate body parts. For example, an orientation of the support body 2 that can be modified compared with a reference orientation allows for (yet) more relaxing or relaxed support of a body part. A corresponding mounting-portion element 18 may, as shown in FIGS. 6-8, e.g. have a monogonal, polygonal, circular, round or pointed geometry. Likewise, movably mounting the support body 2 can provide training options, e.g. for building muscle, since by moving the support body 2 relative to the surface or the base, certain movement sequences associated with a training effect can be performed.

A corresponding mounting-portion element 18 may also be designed as a separate component and, when required, is or can be connected, optionally in a detachable manner (without being damaged or destroyed), to the support body 2 so as to form the support device 1. A corresponding, e.g. spherical, hemispherical, cylindrical or semi-cylindrical or pyramidal mounting-portion element 18 may e.g. be connected to the support body 2 by a hook-and-loop fastener system or an interchangeable fastener system.

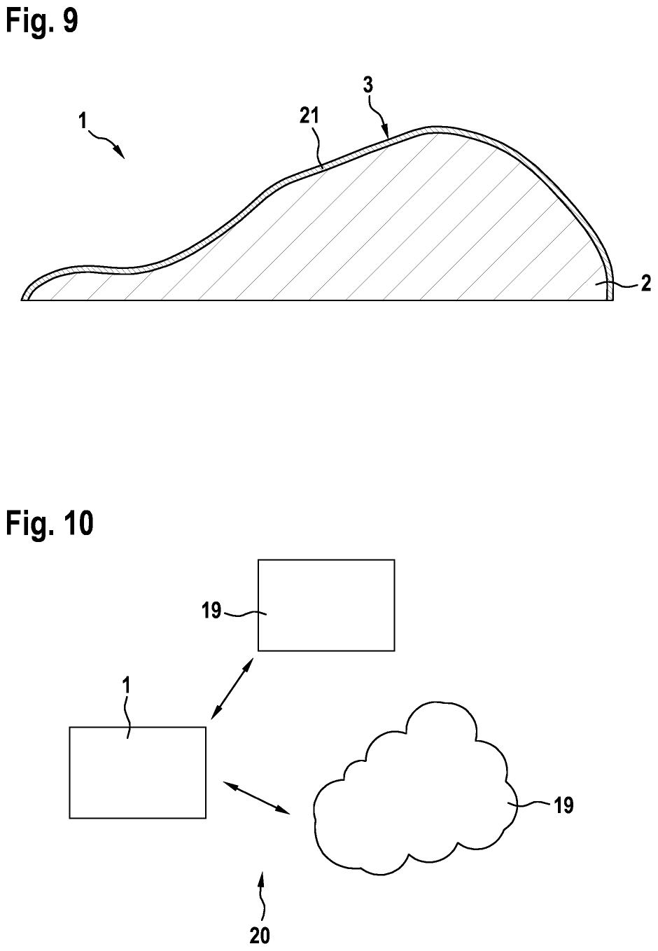

FIG. 9 is a schematic side view of a support device 1 for supporting a body part (not shown) of a user according to an embodiment.

With reference to FIG. 9, it is clear that the support device 1 may comprise a support element 21, which is or can be supported on the support region 3, in particular detachably (without being damaged or destroyed), so as to cover said support region at least in portions, optionally completely. A corresponding support element 21 may in particular be expedient from a hygiene viewpoint, since the support region 3 or the support body 2 is protected against direct contact with the relevant body part and thus e.g. against deposits of skin, sweat, etc. A corresponding support element 21 may be made of a resiliently flexible material or a resiliently flexible material structure, such that the support element 21 does not impair the fit of the support region 3. A corresponding support element 21 may be made of an optionally washable, natural and/or synthetic fabric material or a natural and/or synthetic fabric material structure. Merely by way of example, reference is made to leather or plastics materials.

Although not shown in the drawings, the support device 1 may comprise a fixing device, which is or can be assigned to the support body 2 and/or the support region 3, for temporarily fixing a body part supported on the support region 3, in particular in an intended support position of the body part on the support region 3. Corresponding fixing may facilitate and maintain a desired orientation of a body part supported on the support region 3. In this way, a relaxation effect or therapeutic effect that can be produced by the support device 1 can be assisted. A corresponding fixing device can make it possible to fix a relevant body part directly to the support body 2 or the support region 3; this may e.g. be implemented by a fixing element that is or can be attached to the support body 2 or the support region 3, such as a strap, a loop, etc., which surrounds the relevant body part at least in portions, optionally completely, after being put on, such that the body part is fixed to the support body 2 or the support region 3. A tube-like or tubular fixing device is also conceivable, i.e. a fixing tube made of a fabric, for example, which can be pulled over the support device and the body part that is supported or is to be supported thereon and, in this way, a desired orientation of a body part supported on the support region is facilitated and maintained. A fixing device is also conceivable which makes it possible to fix a superordinate body part, i.e. an arm, for example, in a particular orientation when the body part to be fixed is a hand.

It is applicable to all the embodiments that the support region 3 and/or the support body 2 is made of or comprises at least one magnetisable or magnetic material. Magnetisable or magnetic materials, such as corresponding ferromagnetic metals, may have a positive effect on the blood flow through the corresponding body part, and this can promote therapeutic effects. Magnetisable or magnetic materials may be provided as a solid material or in the form of particles.

It is also applicable to all the embodiments that the support region 3 and/or the support body 2 is, alternatively or additionally, made of or comprises at least one antibacterial material. Antibacterial materials, such as antibacterial metals, may have a positive effect on the hygienic properties of the support region 3 and/or the support body 2. Antibacterial materials may be provided as a solid material or in the form of particles.

With regard to the production of the support device 1, i.e. the support body 2 and/or the support region 3, in principle any manufacturing methods, in particular moulding and/or shaping manufacturing methods, are taken into consideration.

In particular, both additive (build-up) manufacturing methods and subtractive (material-removing) manufacturing methods, i.e. in particular machining manufacturing methods, are taken into consideration.

Additive manufacturing methods may be particularly expedient, since these are particularly suitable for producing corresponding, individually configured geometric-structural designs. Therefore, additive manufacturing methods can be taken into consideration which allow for the additive build-up of three-dimensional objects by selectively hardening, in layers, an optionally powdered construction material that can be selectively hardened in layers (this may e.g. be a metal or a plastics material) by means of radiation energy, i.e. in particular by means of at least one energy beam. Merely by way of example, in this connection reference is made to binder-jetting methods, selective laser-melting methods, selective laser-sintering methods or sterolithography methods.

FIG. 10 is a schematic view of a communication system 20 according to an embodiment. The communication system 20 comprises at least one support device 1, which comprises a transmission device 8, which is configured to transmit a piece of information to at least one communication partner 19 and/or to receive a piece of information transmitted by at least one communication partner 19, and at least one communication partner 19, which is configured to receive a piece of information transmitted by the at least one support device 1 and/or to transmit a piece of information to the at least one support device 1.

The communication partner 19 may e.g. be a mobile terminal, such as a portable computer (laptop), a smartphone, a tablet, or smart glasses, may be a stationary terminal, such as a stationary computer (desktop, server), may be a network application, or may be network storage (cloud), etc.

FIG. 10 is a schematic view of a method for producing a support device 1 for receiving a body part of a user according to an embodiment.

The method serves to produce a support device 1 for supporting at least one body part of a user at least in portions, optionally completely, comprising a support body 2, which comprises at least one support region 3 for supporting at least one body part of a user at least in portions, optionally completely.

The method comprises the step S1, i.e. providing data describing at least one body part of a user that is to be supported on the support region 3 of the support device 1 to be produced at least in portions, optionally completely, and the step S2, i.e. producing at least the support region 3 on the basis of the data such that the support region 3 has, at least in portions, optionally completely, a geometric-structural design that is individually configured on the basis of the data.

The method may further comprise a step S1′, i.e. recording data describing at least one body part of a user that is to be supported on the support region 3 at least in portions, optionally completely. Recording corresponding data that corresponds to the subsequent provision may therefore be part of the method.

The support region 3 and/or the support body 2 may in particular be produced by means of an additive manufacturing method.

Individual features, multiple features or all the features disclosed in connection with a particular embodiment may be combined with individual features, multiple features or all the features disclosed in connection with at least one other embodiment.

1. A support device (1) for supporting at least one body part of a user at least in portions comprising:
a support body (2), which comprises at least one support region (3) for supporting at least one body part of a user at least in portions, wherein
at least the support region (3) has, at least in portions, geometric-structural design that is individually configured on the basis of the data describing at least one body part of a user that is to be supported on the support region (3) at least in portions.

2. The support device according to claim 1, wherein the support region (3) comprises one or more individually configured support-region portions (5), which are designed as receiving portions (4) and are configured to receive a particular body part of a body part that is supported or is to be supported on the support region (3).

3. The support device according to claim 1, comprising a detection device (7), integrated in the support region (3) and/or the support body (2), configured to detect at least one one of a chemical and physical variable of at least one of the support region (3), the support body (2), and a body part supported on the support region (3).

4. The support device according to claim 3, comprising a transmission device (8), which is or can be assigned to the detection device (7), integrated in at least one of the support region (3) and the support body (2), and configured to transmit a piece of detection information describing the at least one of the chemical and physical variable detected by means of the detection device (7) to at least one communication partner (19).

5. The support device according to claim 1, comprising a detection device (9), integrated in at least one of the support region (3) and the support body (2), configured to detect a body part supported on the support region (3).

6. The support device according to claim 5, comprising a transmission device (8), which is or can be assigned to the detection device (9), integrated in the at least one of the support region (3) and the support body (2), and configured to transmit a piece of detection information describing a body part supported on the support region (3) detected by means of the detection device (9) to at least one communication partner (19).

7. The support device according to claim 1, comprising a detection device (10), integrated in the at least one of the support region (3) and the support body (2), configured to detect at least one of a blood pressure, an oxygen saturation, and a pulse, of a user supporting a body part on the support region (3).

8. The support device according to claim 7, comprising a transmission device (8), which is or can be assigned to the detection device (10), integrated in the at least one of the support region (3) and the support body (2), and configured to transmit a piece of detection information describing the at least one of the blood pressure, the oxygen saturation, and the pulse detected by means of the detection device (10) to at least one communication partner (19).

9. The support device according to claim 1, comprising a detection device (11), integrated in the at least one of the support region (3) and the support body (2), configured to detect at least one of a movement of the support body (2) in at least one degree of freedom, relative to a surface and a movement of a body part supported on the support region (3) in at least one degree of freedom, relative to the support body (2).

10. The support device according to claim 9, comprising a transmission device (8), which is or can be assigned to the detection device (11), is integrated in the at least one of the support region (3) and the support body (2), configured to transmit at least one of the movement, detected by means of the detection apparatus (11), of the support body (2) in at least one degree of freedom, relative to a surface and the movement, detected by means of the detection apparatus (11), of a body part supported on the support region (3) in at least one degree of freedom, relative to the support body (2), to at least one communication partner (19).

11. The support device according to claim 3, comprising a storage device (12), integrated in the at least one of the support region (3) and the support body (2), is configured to store at least one piece of detection information detected by means of the detection device (7, 9, 10, 11).

12. The support device according to claim 1, comprising a signal-output device (13), integrated in the at least one of the support region (3) and the support body (2), configured to generate at least one of an acoustic, an optical, and a haptic output signal, on the basis of at least one piece of detection information generated by the or a detection device (7, 9, 10, 11).

13. The support device according to claim 12, wherein the at least one of the acoustic, the optical, and the haptic output signal contains at least one piece of information relating to at least one of a user-side action and an evaluation of a user-side action, on a user, of the support device (1).

14. The support device according to claim 1, wherein the support body (2) is integrated at least in portions in an input apparatus (15) of a device that can be controlled by the input apparatus (15).

15. The support device according to claim 1, comprising a temperature-control device (16), integrated in the at least one of the support region (3) and the support body (2), for controlling at least one of the temperature of the support region (3) and the support body (2) at least in portions.

16. The support device according to claim 15, wherein the temperature-control device (16) comprises a temperature-control duct structure comprising a temperature-control duct through which a temperature-control medium can flow.

17. The support device according to claim 16, wherein the temperature-control device (16) comprises at least one electrically operated temperature-control element.

18. The support device according to claim 14, wherein the temperature-control device (16) comprises at least one temperature-control element, which performs an exothermic chemical reaction after activation.

19. The support device according to claim 1, comprising a fixing device, which is or can be assigned to at least one of the support body (2) and the support region (3), for temporarily fixing a body part supported on the support region (3) in an intended support position of the body part on the support region (3).

20. The support device according to claim 1, wherein the support body (3) comprises a mounting portion (17) for mounting the support body (2) on a surface.

21. The support device according to claim 20, wherein the mounting portion (17) comprises at least one mounting-portion element (18) which lies in a non-planar manner on a planar surface and makes it possible to mount the support body (2) that is mounted on the surface relative to the surface so as to be pivotally movable, in at least one degree of freedom.

22. The support device according to claim 1, wherein the support region (3) is a component that is separate from the support body (2) and can be connected to the support body (2) so as to form the support device (1).

23. The support device according to claim 1, comprising at least one support element (21), which is detachabley supported on the support region (3) so as to cover said support region at least in portions.

24. The support device according to claim 1, wherein the at least one of the support region (3) and the support body (2) comprises at least one magnetisable or magnetic material.

25. The support device according to claim 1, wherein the at least one of the support region (3) and the support body (2) comprises at least one antibacterial material.

26. The support device according to claim 1, wherein the at least one of the support region (3) and the support body (2) is additively manufactured at least in portions.

27. A communication system (20) comprising:
at least one support device (1) according to claim 1, which comprises a transmission device (8), configured to transmit a piece of information to at least one communication partner (19) and/or to receive a piece of information transmitted by at least one communication partner (19), and
at least one communication partner (19), which is configured to receive a piece of information transmitted by the at least one support device (1) and/or to transmit a piece of information to the at least one support device (1).

28. A method for producing a support device (1) according to claim 1, comprising the steps of:
providing data describing at least one body part of a user that is to be supported on the support region (3) of the support device (1) to be produced at least in portions, and
producing at least the support region (3) on the basis of the data such that the support region (3) has, at least in portions, a geometric-structural design that is individually configured on the basis of the data.
